(19) United States
(12) Patent Application Publication
(10) Pub. No.: US 2022/0409894 A1
(43) Pub. Date: Dec. 29, 2022

(54) MODULATION OF VASCULAR ENDOTHELIAL GROWTH FACTOR (VEGF) AND PULSE WIDTH UTILIZATION

(71) Applicant: Leonhardt Ventures LLC, Mission Viejo, CA (US)

(72) Inventors: Howard J. Leonhardt, Mission Viejo, CA (US); John Marchetto, Weston, FL (US)

(21) Appl. No.: 17/809,220

(22) Filed: Jun. 27, 2022

Related U.S. Application Data
(60) Provisional application No. 63/215,841, filed on Jun. 28, 2021.

(51) Int. Cl. A61N 1/32

(52) U.S. Cl. CPC A61N 1/326

(57) ABSTRACT

Described is precise bioelectrical stimulation of a subject's cellular tissue with a selected bioelectric signal to modulate the expression of a peptide by the cellular tissue. More specifically, the application relates to a device that delivers a programmed bioelectric signal or signals, and associated methods for the controlled modulation of a peptide, such as vascular endothelial growth factor (VEGF) and/or hypoxia-inducible factor 1-alpha (HIF1α), or manipulation of stem cells, via precise bioelectrical signals and sequences useful in, for example, orthodontic and other procedures. The bioelectric signals have preferably been further optimized by selecting a desired pulse width for the peptide(s) to be expressed.

### CROSS-REFERENCE TO RELATED APPLICATION(S)

The application claims the benefit under 35 U.S.C. § 119 of U.S. Provisional Patent application serial number U.S. Ser. No. 63/215,841, filed Jun. 28, 2021, the contents of which are incorporated herein by this reference.

### TECHNICAL FIELD

The application relates generally to the field of medical devices and associated methods and treatments, and more specifically to precise bioelectrical stimulation of a subject's cellular tissue. More specifically, the application relates to a device having programmed bioelectric signals, and associated methods for the controlled modulation of a peptide, such as vascular endothelial growth factor (“VEGF”) and/or hypoxia-inducible factor 1-alpha (“HIF-1α”), or manipulation of stem cells, via precise bioelectrical signaling sequences useful in, for example, orthodontic and other procedures. The bioelectric signals have preferably been further optimized by selecting a desired pulse width for the peptide(s).

### BACKGROUND

Vascular endothelial growth factor (“VEGF”) is a signaling protein that promotes the growth of new blood vessels. VEGF forms part of the mechanism that restores the blood supply to cells and tissues when they are deprived of oxygenated blood due to compromised blood circulation.

Kanno et al. “Establishment of a Simple and Practical Procedure Applicable to Therapeutic Angiogenesis” Circulation 99(20):2682-2687 (1999) described that when cultured skeletal muscle cells were electrically stimulated at a voltage that did not cause their contraction, VEGF mRNA was augmented at an optimal-frequency stimulation. This increase of VEGF mRNA was derived primarily from transcriptional activation. Electrical stimulation increased the secretion of VEGF protein into the medium. This conditioned medium then augmented the growth of endothelial cells. The effect of electrical stimulation was further confirmed in a rat model of hind limb ischemia. The tibialis anterior muscle in the ischemic limb was electrically stimulated. The frequency of stimulation was 50 Hz and strength was 0.1 V, which was far below the threshold for muscle contraction. After a 5-day stimulation, there was a significant increase in blood flow within the muscle. Immunohistochemical analysis revealed that VEGF protein was synthesized and capillary density was significantly increased in the stimulated muscle. See, also, Spadaccio et al. “In Situ Electrostimulation Drives a Regenerative Shift in the Zone of Infarcted Myocardium” Cell Transplantation, Vol. 21, pp. 493-503, 2013 (devices delivered a 3-V pulse at a rate of 10 Hz) and Liebano, R. E., and Perez Machado, A. F. “Vascular Endothelial Growth Factor Release Following Electrical Stimulation in Human Subjects.” Advances in Wound Care Vol. 3, 2 (2014): 98-103. doi: 10.1089/wound.2013.0427.

U.S. Pat. No. 10,960,206 to Leonhardt et al. (Mar. 30, 2021) for “Bioelectric Stimulator”, the contents of which are incorporated herein by this reference, discloses (e.g., in FIG. 19) that expression of VEGF is upregulated by the application of a 50 Hz, square wave bioelectric signal to a subject's tissue. See, also U.S. Pat. No. 6,988,004 to Kanno et al. (Jan. 17, 2006) and U.S. Pat. No. 5,817,139 to Kasano, the contents of each of which are incorporated herein by this reference.

In Kanno et al. “Establishment of a Simple and Practical Procedure Applicable to Therapeutic Angiogenesis” Circulation, 99:2682-2687 (1999), the contents of which are incorporated herein by this reference, “[e]lectrical stimulation was started on the day after electrode implantation and continued for 5 days with a 0.3-ms stimulus width, 50-Hz stimulus frequency, and 0.1-V stimulus strength, which was far below the threshold of TA muscle contraction.”

Other bioelectric signals for VEGF and other peptides are described in, for example, the incorporated U.S. Pat. No. 10,960,206; US 20200330753 A1 to Leonhardt, Marchetto et al. for “Orthodontic treatment”, published on Oct. 22, 2020; US 20190022389 A1 to Leonhardt et al. for “Method of Treating Inflammation”; US 20200324106 A1 to Leonhardt et al. (Oct. 15, 2020) for “Bioelectric Stimulation for Sonic Hedgehog Expression”; US 20200289826 A1 to Leonhardt et al. (Sep. 17, 2020) for “Klotho Modulation”; and U.S. 2020/033079 A1 to Leonhardt et al. for “Bioelectric OPG Treatment of Cancer”, the contents of each of which are incorporated herein by this reference.

### BRIEF SUMMARY

Heretofore, it has been assumed that if the frequency of the applied bioelectric signal were correct and the voltage were sufficient to overcome resistance to reach the target cellular tissue, that small variations in pulse width or duration would not affect results in modulating protein expression.

As described herein however, we tested this hypothesis and determined the effects on protein expression by a target cellular tissue when a bioelectric signal having a constant frequency, voltage, and amperage was applied to the tissue, and only the duration of the pulse width was modified.

Described herein is that protein expression (e.g., as measured by mRNA expression by RT-qPCR) can be further modulated (and even completely change from upregulation to downregulation) by changing the duration of the pulse width of the bioelectric signal applied to the target cellular tissue.

Thus, described is an improvement in a method of modulating the expression of a peptide by a target cellular tissue of the type comprising applying a bioelectric signal to the target cellular tissue for a sufficient time to modulate (upregulate or downregulate) mRNA expression for the peptide, the method comprising: selecting a bioelectric signal with a frequency particular for modulating the peptide; determining a desired pulse width of the selected bioelectric signal for expression of the mRNA encoding the peptide; and applying the bioelectric signal to the target cellular tissue so as to modulate expression of the peptide in the target cellular tissue.

Determining a desired pulse width of the selected bioelectric signal for expression of the mRNA encoding the peptide typically comprises measuring relative mRNA expression for the peptide (e.g., by use of Reverse Transcription Quantitative PCR (“RT-qPCR”)) utilizing different bioelectric signals having different pulse widths.

With such a method, the peptide may be selected from the group consisting of Activin B, bone morphogenic protein 9 (BMP9), COL17A1, C-X-C motif chemokine 5 (CXCL5), epidermal growth factor (EGF), follistatin, hepatocyte growth factor (HGF), hypoxia-inducible factor 1-alpha (HIF-1α), insulin-like growth factor 1 (IGF-1), interleukin-6 (IL-6), klotho, osteoprotegerin (OPG), platelet-derived growth factor (PDGF), receptor activator of nuclear factor kappa-B ligand (RANKL), stromal cell-derived factor 1 (SDF1), Sonic Hedgehog, Transforming growth factor beta 1 (TGF-β1), tissue necrosis factor (TNF), and vascular endothelial growth factor (VEGF).

With such a method, the frequency is typically selected from the group consisting of 1 Hz, 5 Hz, 10 Hz, 15 Hz, 20 Hz, 22 Hz, 25 Hz, 30 Hz, 50 Hz, 80 to 100 Hz, 100 Hz, 150 Hz, 300 Hz, 500 Hz, 2,000 Hz, 3 MHz, 2/100 Hz (frequency modulated biphasic signal), 50 Hz to 100 Hz (frequency modulated biphasic signal), and any combination thereof.

Described further herein is a bioelectric stimulator programmed to produce at least one bioelectric signal that upregulates or downregulates the expression of a peptide (such as VEGF) in a mammalian target tissue.

In certain embodiments, the bioelectric stimulator is programmed to produce at least one bioelectric signal that upregulates the expression of VEGF in the target tissue. In certain embodiments, the bioelectric stimulator is programmed to produce at least one bioelectric signal that downregulates the expression of VEGF in the target tissue. Upregulation of the expression of VEGF is useful for loosening up bone to reform dental arches, teeth, etc. for re-aligning the teeth (e.g., by increasing local capillary proliferation to increase blood flow which enhances the removal of inorganic components to soften the bone). Upregulating expression of VEGF may also be used to stabilize the teeth in correct position after re-alignment, e.g., by increasing local capillary proliferation, which increases local blood flow thus enhancing the supply to the bone of inorganic components needed to harden the bone. The described system can accelerate tooth movement in an orthodontic procedure by up to 70%.

A bioelectric stimulator programmed to produce a bioelectric signal that upregulates or downregulates VEGF in a target tissue, wherein the bioelectric signal has a pulse width of (a) from about 50 microseconds (“μsec”) to about 200 μsec or (b) from about 400 μsec to about 10,000 μsec. Another such bioelectric signal that downregulates VEGF has a frequency of (within 15%) 30 Hz and a voltage of 3.5 mV as may be measured at the level of the stimulated cell(s).

In certain such embodiments, the bioelectric signal has a pulse width from about 50 μsec to about 200 μsec.

In certain such embodiments, the bioelectric signal has a pulse width from greater than 400 μsec to about 10,000 μsec.

In certain such embodiments, the bioelectric stimulator upregulates VEGF in the target tissue.

In certain such embodiments, the bioelectric signal has a pulse width of 100 μsec.

In certain such embodiments, the bioelectric signal has a pulse width of about 500 μsec.

In certain such embodiments, the bioelectric signal has a pulse width of about 10,000 μsec.

In certain such embodiments, the bioelectric stimulator downregulates VEGF in the target tissue.

In certain such embodiments, the bioelectric signal has a pulse width of about 2,000 μsec.

In certain such embodiments, the bioelectric stimulator is programmed to produce a plurality of bioelectric signals.

Also described is a method of using a bioelectric stimulator to stimulate cellular tissue to modulate the expression of vascular endothelial growth factor (“VEGF”) in the tissue, the method comprising: using the bioelectric stimulator to apply a bioelectric signal to the tissue, wherein the bioelectric signal(s) has a pulse width of (a) from about 50 microseconds (“μsec”) to about 200 μsec or (b) from about 400 μsec to about 10,000 μsec.

In certain such embodiments, the tissue is selected from the group consisting of bone, dental arch, dental gum tissue, and any combination(s) thereof.

Also described is a method of modulating expression of VEGF in a cell, the method comprising: applying to the cell a bioelectric signal that upregulates or downregulates the expression of VEGF wherein the bioelectric signal(s) has a pulse width of (a) from about 50 microseconds (“μsec”) to about 200 μsec or (b) from about 400 μsec to about 10,000 μsec.

In certain such embodiments, the bioelectric signal upregulates the expression of VEGF in the cell.

In certain such embodiments, the bioelectric signal has a pulse width of 100 μsec.

In certain such embodiments, the bioelectric signal has a pulse width of about 500 μsec.

In certain such embodiments, the bioelectric signal has a pulse width of about 10,000 μsec.

In certain such embodiments, the bioelectric signal downregulates VEGF in the target tissue.

In certain such embodiments, the bioelectric signal has a pulse width of about 2,000 μsec.

In certain embodiments, the bioelectric stimulator upregulates VEGF in the target cellular tissue and the bioelectric signal has a frequency selected from the group consisting of 25 Hz, 50 Hz, 75 Hz, 100 Hz, 500 Hz, 750 Hz, and 1,000 Hz.

In certain embodiments, the bioelectric stimulator upregulates hypoxia-inducible factor 1-alpha (HIF-1α) in the target cellular tissue and the bioelectric signal has a frequency selected from the group consisting of 25 Hz, 50 Hz, 75 Hz, and 100 Hz. HIF-1α is a master transcriptional regulator of an array of genes involved in angiogenesis, autophagy, cell survival, and glucose metabolism. Some of the useful properties of the signaling of HIF-1α include promoting heart regeneration, retinal eye regeneration, and muscle regeneration. VEGF is an injury-induced HIF-1α target gene.

In certain embodiments, the bioelectric stimulator downregulates VEGF in the target tissue and the bioelectric signal has a frequency of about 250 Hz.

Bioelectric signals can be delivered using a constant current or a constant voltage delivery method. Constant current delivery typically ranges from 100 μA to 50 mA. If the skin is contacted, bioelectric signals are typically allowed to increase to the point that a somatosensory response is reported by the patient. Constant voltage delivery would typically range from 1 mV to 20 V/cm. These ranges can vary, dependent upon the resistance of the cellular tissue to be treated.

In certain embodiments, the bioelectric stimulator is programmed to produce further bioelectric signals, which aid the subject.

The descried bioelectric stimulator may be used to stimulate tissue of a subject, the method comprising: connecting the bioelectric stimulator to the target tissue of the subject, and actuating the bioelectric stimulator to produce the programmed bioelectric signal(s).

A biostimulator and/or associated methods, as described herein, may be for use in an orthodontic procedure. In such a case, typically, the tissue is dental gum, bone or the dental arch of a subject in need thereof.

Vascular endothelial growth factor (VEGF or VEGF-A) is useful for growing new mature blood vessels and improving blood circulation and have applications in treating conditions such as wound healing, stroke recovery, hearing recovery, erectile dysfunction, and hair loss. It is also useful for facilitating tooth movement during orthodontic procedures.

### BRIEF DESCRIPTION OF THE DRAWINGS

FIG. 1 is an exemplary electric signal generator.

FIG. 2 is an intraoral device having electrodes for applying bioelectric signals to the gums of a subject.

FIG. 3 is Table 1, a one-sample T-Test of pulse width (pec), frequency, and volts of mouse osteoblast stimulated at various conditions

FIG. 4 is a graph depicting mRNA expression of VEGF-A of osteoblast stimulated at 1V, 50 Hz, and 50-20,000 μsec.

FIG. 5 depicts a model bioelectric stimulation system.

FIG. 6 is a graph depicting HIF-1alpha and VEGF-A mRNA expression in porcine heart due to bioelectric stimulation (bioelectric signal application0.

### DETAILED DESCRIPTION

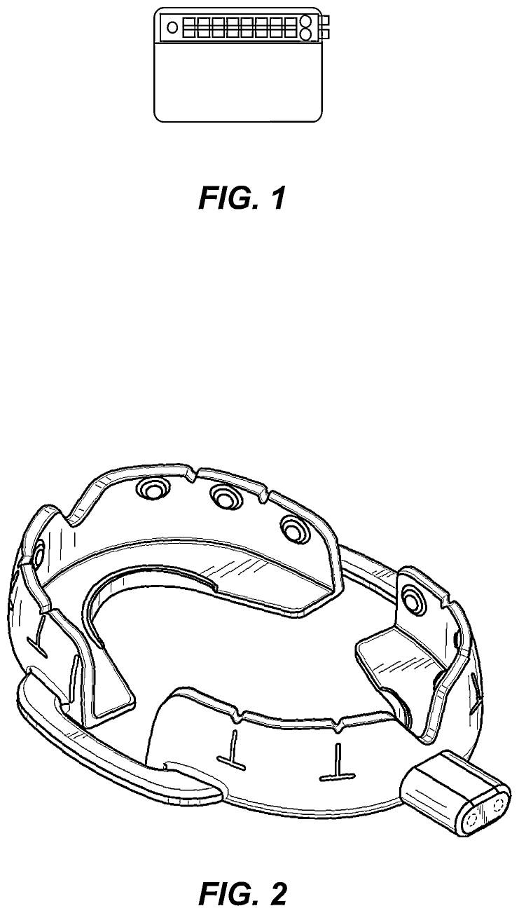

Referring now to FIG. 1, depicted is a biostimulator for use in the treatment of a, for example, human subject. A micro voltage signal generator for use herein may be produced utilizing the same techniques to produce a standard heart pacemaker well known to a person of ordinary skill in the art. Programmable microvoltage generator are commercially available.

A micro voltage signal generator for use herein may be produced utilizing the same techniques to produce a standard heart pacemaker well known to a person of ordinary skill in the art. An exemplary microvoltage generator is available from Mettler Electronics Corp. of Anaheim, Calif., US or HTM Electrônica of Amparo, BR. The leading pacemaker manufacturers are Medtronic, Boston Scientific Guidant, Abbott St. Jude, BioTronik and Sorin Biomedica.

Construction of the electric signal generators and pacemakers, are known in the art and can be obtained from OEM suppliers as well as their accompanying chargers and programmers. The electric signal generators are programmed to produce specific signals to lead to specific protein expressions at precisely the right time for, e.g., optimal treatment or regeneration.

Electrodes may be used to deliver a bioelectric signal sequence to the subject's cellular tissue (e.g., skin, gums, or muscle).

In use, such electrodes are connected to the subject's body and placed for administration of the described bioelectric signal sequence.

The biostimulator of FIG. 1 may be used with, for example, multiple soft conductive electrode pads to apply the bioelectric signal to the target tissue. Electrodes may be used to deliver a bioelectric signal to the subject. In certain embodiments, other electrodes that may be used to deliver bioelectric signals are depicted in the incorporated US 20200330753 A1 to Leonhardt, Marchetto et al.

As depicted in FIGS. 1 and 2 and the incorporated US 20200330753 A1, a system useful in an orthodontic procedure comprises a bioelectric stimulator (FIG. 1) programmed to produce sequential electrical signals in electrical association with (e.g., via stem portion) a mouth piece comprised of a polymer and constructed to fit about and/or over the subject's teeth, braces or clear aligners and in proximity of the subject's gums.

The bioelectric signal generator (FIG. 1) is used to generate the specific bioelectric signal(s) typically transmitted via electrodes (e.g., contact pins/points) on the mouthpiece (FIG. 2) that cause the specific cells and proteins to be expressed by the cells associated with the gums and bone. The bioelectric stimulator is programmed with selected signals in a designed sequence to facilitate bone resorption/demineralization (softening). In the depicted embodiment, the bioelectric stimulator sends preprogrammed bioelectric signals via the intraoral electrode mouthpiece during an orthodontic procedure to the subject's gum and bone tissue.

The biostimulator is actuated and runs through programmed signals to modulate the production of a bioelectric signal or signals that can induce a subject to increase or decrease the expression of a peptide for delivery to the subject. For an orthodontic procedure, the peptides typically include RANKL, VEGF, and OPG.

A particularly preferred bioelectric signal for upregulating the expression and/or release of VEGF in mammalian cellular tissue has a frequency of 50 pulses per second at a pulse width of 500 μseconds in a continuous delivery mode.

In addition to the bioelectric signals identified herein for VEGF, the frequencies of the bioelectric signals typically associated with various peptides are:
1. Activin B (150 Hz),
2. bone morphogenic protein 9 (BMP9) (100 Hz or 300 Hz),
3. COL17A1 (25 Hz upregulate) (50 Hz downregulate),
4. C-X-C motif chemokine 5 (CXCL5) (1 Hz),
5. epidermal growth factor (EGF) (500 Hz), follistatin (50 Hz, then 100 Hz),
6. hepatocyte growth factor (HGF) (50 Hz),
7. hypoxia-inducible factor 1-alpha (HIF-1α) (80 to 100 Hz),
8. insulin-like growth factor 1 (IGF-1) (22 Hz),
9. interleukin-6 (IL-6) (50 Hz),
10. klotho (20 Hz),
11. osteoprotegerin (OPG) (3 MHz or 2,000 Hz),
12. platelet-derived growth factor (PDGF) (10 Hz or 100 Hz),
13. receptor activator of nuclear factor kappa-B ligand (RANKL) (2/100 Hz frequency modulated biphasic signal),
14. stromal cell-derived factor 1 (SDF1) (100 Hz),
15. Sonic Hedgehog (a biphasic pulse at from 50 Hz to 100 Hz; 5 Hz, 10 Hz, 20 Hz, 50 Hz, 100 Hz),
16. stem cell differentiation (20 Hz),
17. tissue necrosis factor (TNF) (2/100 Hz, alternating frequency, followed by 15 Hz, followed by 30 Hz),
18. tropoelastin (50 Hz), and
19. Transforming growth factor beta 1 (TGF-β1) (≥75 Hz).

The expression of transforming growth factor beta 1 (TGF-β1) is upregulated by a square, biphasic waveform at 50% duty, wherein the frequency is at least 75 Hz and the signal amplitude is, e.g., 1.0 V.

As used herein with respect to the frequency of a particular bioelectric signal, the term “about” means within 15% plus or minus.

As used herein with respect to the pulse width of a particular bioelectric signal, the term “about” means within 33% plus or minus.

Typical subjects to be treated are mammals such as humans.

In certain embodiments, the bioelectric stimulator is programmed to produce further bioelectric signals, such as those that induce expression by cellular tissue of osteoprotegerin or “OPG”, RANKL, SDF-1, PDGF, a signal for stem cell homing, PDGF, different signals for stem cell proliferation, activin-B, EGF, IGF-1, tropoelastin, VEGF, follistatin, HGF, TGF-β1, and any combination thereof.

For example in orthodontic procedures, upregulation of VEGF allows for an increase in the rate of tooth movement. Preferably, the process increases blood flow during active tooth movement of the teeth (e.g., RANKL is a key initiator), and the inorganic components of bone are able to be moved away from the compression side of the tooth, thus allowing the tooth to move. Conversely, when bone needs to be deposited, increased blood flow carries the inorganic components back into the area in need of stabilization.

The length of time that a bioelectric signal for VEGF is applied to the subject for a single treatment in a treatment regimen can vary dependent on, for example, the disease or condition being treated. For example, in assisting orthodontic movement of teeth, the length of time of application will typically be between 2 and 15 minutes (e.g., 5 minutes continuously) per application to the cellular tissue. For example, in treating stroke the length of time of application will typically be between 5 and 15 minutes (e.g., 10 minutes) per application. In treating erectile dysfunction, the length of time of application will typically be between 2 and 20 minutes (e.g., 5, 12, or 15 minutes) per application. In aiding hearing recovery, the length of time of application will typically be between 2 and 10 minutes (e.g., 5 minutes) per application. For pain reduction (e.g., in dental applications), the length of time of application will typically be between 2 and 10 minutes (e.g., 5 minutes) per application. In assisting hair growth, the length of time of application will typically be between 2 and 15 minutes (e.g., 5 minutes continuously) per application. In enhancing skin appearance, the length of time of application of the bioelectric signal to cellular tissue will typically be between 2 and 15 minutes (e.g., 5 minutes continuously) per application.

The invention is further described with the aid of the following illustrative Examples.

### EXAMPLES

### Example I—Effects of Pulse Width on MC3T3-E1 Cells on VEGF-A mRNA Expression

Purpose: The purpose of this study was to analyze the effects of pulse width of mouse osteoblast (MC3T3-E1) cells stimulated at 1V, 50 Hz, 50-20,000 μsec with RT-qPCR.

Methods: Mouse osteoblast cells were cultured and stimulated using pulse width ranging from 50 to 20,000 μsec at 1 V, 50 Hz for 30 minutes using a constant voltage waveform generator RIGOL. Gene expression was analyzed by extracting mRNA from osteoblasts and applying RT-qPCR assessment to quantify VEGF-A mRNA expression.

Electrical Signals: MC3T3-E1 cells were stimulated for 30 minutes at 1V, 50 Hz, 50-20,000 μsec with the RIGOL stimulator.

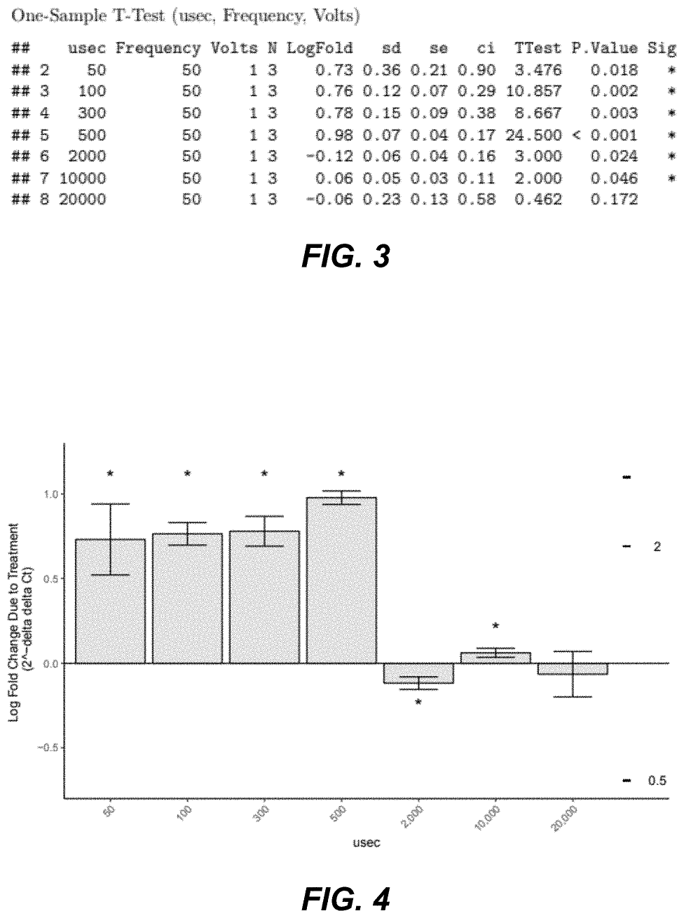

Results: Table 1 is depicted in FIG. 3, which is a one-sample T-Test on μsec, frequency, and volts of mouse osteoblast stimulated at various conditions. See, also, FIG. 4.

Conclusions: As described herein, pulse width plays a significant role on VEGF-A mRNA expression. mRNA expression was significantly, and substantially increased with pulse-width levels ranging from 50-500 μsec. At 10,000 μsec, expression increases were significant, albeit relatively small, while 2,000 μsec produced significant, but again small decreases in expression. Therefore, pulse width plays a major role in gene expression during bioelectric stimulation.

### Example II—Modulation of VEGF-A and Hypoxia-Inducible Factor 1-Alpha (HIF1α)

Purpose: Investigated was a spectrum of noninvasive bioelectric stimulation (BES) parameters that would modulate HIF-1α and VEGF-A expression in porcine heart tissue.

Electrical Signals: The tissue was stimulated for 30 minutes with a square, biphasic waveform at 50% duty cycle. Frequency and voltages were fixed and set from 5 Hz to 2500 Hz (e.g., 5 Hz, 10 Hz, 25 Hz, 50 Hz, 75 Hz, 100 Hz, 250 Hz, 500 Hz, 750 Hz, 1,000 Hz, 2,500 Hz, and control) and 1.0 V.

Methods: Tissue received from the Midwest Swine Institute was kept in cold, ringer solution and viability of tissue was analyzed with a fluorometric detection method before performing this study. Bioelectric signals were applied to heart tissue in vitro using a commercially available Rigol DG1022Z function generator (Beijing, China), which was further programmed via a 6-well stimulating plate interface (IONOPTIX, Westwood, Mass., US). To induce uniform electric fields in all stimulation chambers, 1.3 mL of DMEM solution was added to each well before BES signal application.

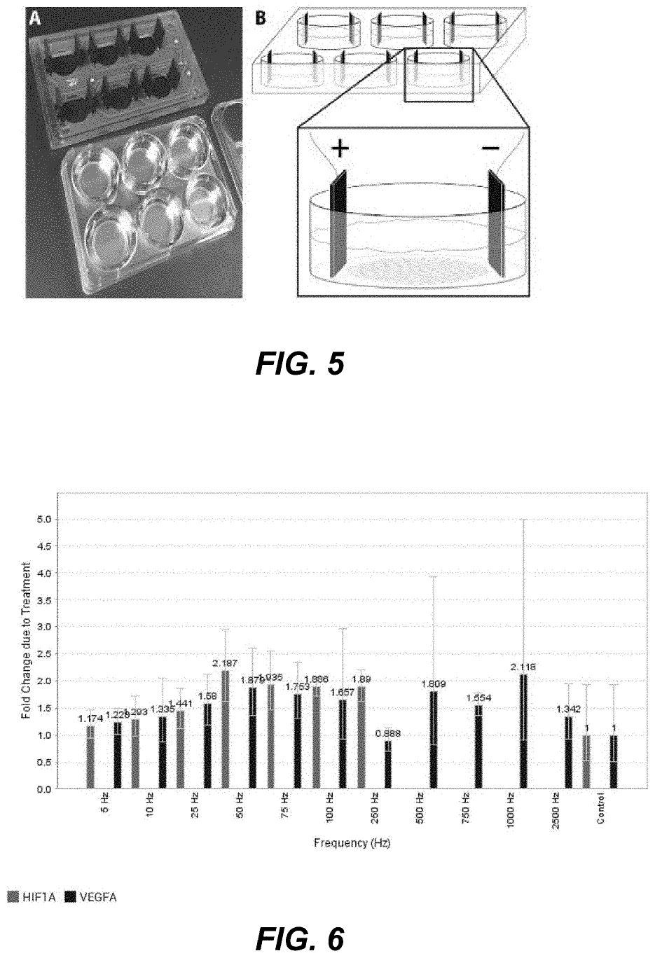

FIG. 5 depicts a bioelectric stimulation system. Cells were placed in each dish and cultured to 80% to 100% confluency. Once confluent, cells were stimulated using an electrode array (shown at the top of panel A of the figure), which was inverted and introduced into the 6-well dish where cells were grown. Each well received uniform stimulation via a pair of carbon electrodes positioned at opposite sides (panel B of FIG. 5.)

Gene expression was determined by homogenizing the tissue, extracting mRNA according to the manufacturer's instructions. RNA quality was determined using a spectrophotometer and was reverse transcribed using a cDNA conversion kit. The cDNA and RT2 SYBR Green qPCR Master mix was used on a Custom RT2 Profiler Array.

The Qiagen data analysis software was used to calculate fold change/regulation using the delta-delta CT method, in which delta CT is calculated between the gene of interest (GOI) and an average of housekeeping genes (HKG), followed by delta-delta CT calculation (delta CT(experiment)-delta CT(control). Fold change is then calculated using the 2\hat{ }(-delta delta CT) formula.

Results: FIG. 6 shows mRNA fold change of HIF-1α and VEGF-A in response to specific stimulation signals. The mRNA expression for HIF-1α had a peak above the threshold at 50 Hz and VEGF-A at 1000 Hz. The expression of VEGF-A decreased to 0.888-fold at 250 Hz. The mRNA expression for HIF-1α had a peak above the threshold at 50 Hz and VEGF-A at 1,000 Hz.

The expression at several frequency conditions was less than 2-fold, which is below the threshold for gene expression. Further experiments would be useful to better optimize BES signals for HIF-1α and VEGF-A.

### Example III—Further Downregulation of VEGF

A bioelectric signal (30 Hz, 3.5 mV) was tested in Retinal Pigment Epithelium (RPE) cells (ATCC), which cells were stimulated for 30 minutes. The expression of genes was assessed on a Qiagen qPCR array (QIAGEN Redwood City, Calif., US). It was found that the stimulation of RPE cells at 30 Hz and signal strength of 3.5 mV, resulted in a decrease in the gene for VEGF as well as for the following genes: stromal cell-derived factor (SDF-1), elastin (ELN), follistatin (FN1), hypoxia-inducible factor-1 alpha (HIF1A), insulin-like growth factor 1 (IGF1), matrix metallopeptidase 9 (MMP9), nitric oxide synthase 1 (NOS1), and transforming growth factor-beta 1 (TGF-B).

### REFERENCES

(The Contents of the Entirety of Each of which is Incorporated Herein by this Reference.)
Beugels et al. “Electrical stimulation promotes the angiogenic potential of adipose-derived stem cells” Sci Rep 9, 12076 (2019). https://doi.org/10.1038/s41598-019-48369-w.
Kanno et al. “Establishment of a Simple and Practical Procedure Applicable to Therapeutic Angiogenesis” Circulation 99(20):2682-2687 (1999).
Liebano, R. E., and Perez Machado, A. F. “Vascular Endothelial Growth Factor Release Following Electrical Stimulation in Human Subjects.” Advances in Wound Care Vol. 3, 2 (2014): 98-103. doi:10.1089/wound.2013.0427.
Liu et al. “Correction of Diabetic Erectile Dysfunction with Adipose Derived Stem Cells Modified with the Vascular Endothelial Growth Factor Gene in a Rodent Diabetic Model” PLoS ONE 8(8): e72790 (2013)://doi.org/10.1371/journal.pone.0072790.
Morimoto et al. “Electrical Stimulation Enhances Migratory Ability of Transplanted Bone Marrow Stromal Cells in a Rodent Ischemic Stroke Model” Cell Physiol Biochem 2018; 46:57-68.
Russold, M. and Jarvis, J. C. “Implantable stimulator featuring multiple programs, adjustable stimulation amplitude and bi-directional communication for implantation in mice” Med. Biol. Eng. Comput. 45(7):695-699 (2007).
Salcedo et al. “Low current electrical stimulation upregulations cytokine expression in the anal sphincter” Int J Colorectal Dis. 2012 February; 27(2):221-5. doi: 10.1007/s00384-011-1324-3.
Spadaccio et al. “In Situ Electrostimulation Drives a Regenerative Shift in the Zone of Infarcted Myocardium” Cell Transplantation, Vol. 21, pp. 493-503, 2013.
U.S. Pat. No. 10,960,206 to Leonhardt et al. for “Bioelectric Stimulator” (Mar. 20, 2021).
US 20200330753 A1 to Leonhardt, Marchetto, et al. for “Orthodontic treatment”, published on Oct. 22, 2020.
US 20190022389-A1 to Leonhardt et al. for “Method of Treating Inflammation”.
US 20200324106 A1 to Leonhardt et al. (Oct. 15, 2020) for “Bioelectric Stimulation for Sonic Hedgehog Expression”.
US 20200289826 A1 to Leonhardt et al. (Sep. 17, 2020) for “Klotho Modulation”.
U.S. Publication No. 2020/033079 A1 to Leonhardt et al. “Bioelectric OPG Treatment of Cancer”.

What is claimed is:

1. A bioelectric stimulator comprising an electric signal generator and electrodes, which electric signal generator is programmed to produce a bioelectric signal that modulates expression and/or release of vascular endothelial growth factor (“VEGF”) in a target cellular tissue, wherein the bioelectric signal has a pulse width of (a) from about 50 microseconds (“μsec”) to about 200 μsec or (b) from about 400 μsec to about 10,000 μsec.

2. The bioelectric stimulator of claim 1, wherein the bioelectric signal has a pulse width from about 50 μsec to about 200 μsec.

3. The bioelectric stimulator of claim 1, wherein the bioelectric signal has a pulse width from greater than 400 μsec to about 10,000 μsec.

4. The bioelectric stimulator of claim 1, wherein the bioelectric stimulator upregulates VEGF in the target cellular tissue and the bioelectric signal has a frequency selected from the group consisting of 25 Hz, 50 Hz, 75 Hz, 100 Hz, 500 Hz, 750 Hz, and 1,000 Hz.

5. The bioelectric stimulator of claim 1, wherein the bioelectric signal has a pulse width of about 100 μsec.

6. The bioelectric stimulator of claim 1, wherein the bioelectric signal has a pulse width of about 500 μsec.

7. The bioelectric stimulator of claim 1, wherein the bioelectric signal has a pulse width of about 10,000 μsec.

8. The bioelectric stimulator of claim 1, wherein the bioelectric stimulator downregulates VEGF in the target cellular tissue and the bioelectric signal has a frequency of about 250 Hz.

9. The bioelectric stimulator of claim 8, wherein the bioelectric signal has a pulse width of about 2,000 μsec.

10. The bioelectric stimulator of claim 1, wherein the bioelectric stimulator is programmed to produce a plurality of bioelectric signals.

11. A method of using the bioelectric stimulator of claim 1 to stimulate cellular tissue to modulate the expression and/or release of vascular endothelial growth factor (VEGF) in the cellular tissue, the method comprising:
using the bioelectric stimulator to apply the programmed bioelectric signal(s) to the cellular tissue, so as to modulate the expression and/or release of VEGF by the cellular tissue.

12. The method according to claim 11, wherein the cellular tissue is selected from the group consisting of bone, dental arch, dental gum tissue, and any combination(s) thereof.

13. A method of treating a cell, the method comprising:
stimulating the cell to express and/or release vascular endothelial growth factor (VEGF) by applying a bioelectric signal to the cell,
wherein the bioelectric signal comprises, within 15%, a square, biphasic waveform at 50% duty cycle, at a frequency selected from the group consisting of 25 Hz, 50 Hz, 75 Hz, 100 Hz, 500 Hz, 750 Hz, and 1,000 Hz and 1.0 V as may be measured at the level of the cell.

14. The method according to claim 13, wherein the bioelectric signal has a pulse width selected from the group consisting of 100 μsec, 500 μsec, and 10,000 μsec.

15. The method according to claim 14, wherein the bioelectric signal has a pulse width of 100 μsec and a frequency of 1,000 Hz.

16. A method of modulating expression of a desired peptide by a target cellular tissue of the type comprising applying at least one bioelectric signal to the target cellular tissue for a sufficient time to modulate mRNA expression of the desired peptide, the method comprising:
selecting a bioelectric signal with a frequency particular for modulating the desired peptide;
determining a desired pulse width of the selected bioelectric signal for expression of the mRNA encoding the desired peptide; and
applying the bioelectric signal to the target cellular tissue at a voltage/amperage sufficient to overcome resistance to safely reach the target cellular tissue
so as to modulate expression of the desired peptide in the target cellular tissue.

17. The method according to claim 16, wherein the desired peptide is selected from the group consisting of Activin B, bone morphogenic protein 9 (BMP9), COL17A1, C-X-C motif chemokine 5 (CXCL5), epidermal growth factor (EGF), follistatin, hepatocyte growth factor (HGF), hypoxia-inducible factor 1-alpha (HIF-1α), insulin-like growth factor 1 (IGF-1), interleukin-6 (IL-6), klotho, osteoprotegerin (OPG), platelet-derived growth factor (PDGF), receptor activator of nuclear factor kappa-B ligand (RANKL), stromal cell-derived factor 1 (SDF1), Sonic Hedgehog, tissue necrosis factor (TNF), vascular endothelial growth factor (VEGF), transforming growth factor beta 1 (TGF-β1), and any combination thereof.

18. The method according to claim 16, wherein the frequency is selected from the group consisting of 1 Hz, 5 Hz, 10 Hz, 15 Hz, 20 Hz, 22 Hz, 25 Hz, 30 Hz, 50 Hz, 75 Hz, 80 to 100 Hz, 100 Hz, 150 Hz, 250 Hz, 300 Hz, 500 Hz, 1,000 Hz, 2,000 Hz, 3 MHz, 2/100 Hz (frequency modulated biphasic signal), 50 Hz to 100 Hz (frequency modulated biphasic signal), and any combination thereof.

19. The method according to claim 16, wherein determining a desired pulse width of the selected bioelectric signal for expression of the mRNA encoding the desired peptide comprises measuring relative mRNA expression associated with the desired peptide utilizing different bioelectric signals having different pulse widths.

20. A method of treating a cell, the method comprising:
stimulating the cell to express and/or release hypoxia-inducible factor 1-alpha (HIF1α) by applying a bioelectric signal to the cell,
wherein the bioelectric signal comprises, within 15%, a square, biphasic waveform at 50% duty cycle, at a frequency selected from the group consisting of 25 Hz, 50 Hz, 75 Hz, and 100 Hz and 1.0 V as may be measured at the level of the cell.
